(19) United States
(12) Patent Application Publication
(10) Pub. No.: US 2005/0038018 A1
(43) Pub. Date: Feb. 17, 2005

(54) Meloxicam compositions

(75) Inventors: Hideyoshi Kanbe, Chiba (JP); Minoru Okada, Chiba (JP); Hiroshi Otaki, Chiba (JP); Norimitsu Umehara, Saitama (JP); Akira Takahashi, Ibaraki (JP); Toshiaki Horie, Katori-gun (JP)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/885,260

(22) Filed: Jul. 6, 2004

Related U.S. Application Data
(60) Provisional application No. 60/489,647, filed on Jul. 24, 2003.
(60) Provisional application No. 60/489,958, filed on Jul. 24, 2003.
(60) Provisional application No. 60/489,646, filed on Jul. 24, 2003.

(30) Foreign Application Priority Data
Jul. 9, 2003 (EP) 03 015 436
Jul. 9, 2003 (EP) 03 015 435
Jul. 10, 2003 (EP) 03 015 741

(51) Int. Cl. A61K 31/54; A61K 33/10; A61K 33/08; A61K 33/12

(52) U.S. Cl. 514/226.5; 424/683; 424/690; 424/687

(57) ABSTRACT

A pharmaceutical composition comprising: (a) meloxicam or a pharmaceutically acceptable salt thereof; and (b) one or more additional pharmaceutically active compounds selected from antacids, sedatives, and central nervous system stimulants, and the use of such composition of an inflammatory disease, symptoms of an inflammatory disease, including various symptoms thereof, and/or headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction and/or cold and various symptoms of cold such as sore throat, chill, pyrexia, headache, arthralgia and muscle pain.

### RELATED APPLICATIONS

This application claims benefit of U.S. Ser. No. 60/489,647, filed Jul. 24, 2003, U.S. Ser. No. 60/489,958, filed Jul. 24, 2003, and U.S. Ser. No. 60/489,646, filed Jul. 24, 2003, and claims priority to European Application No. 03 015 436.3, filed Jul. 9, 2003, European Application No. 03 015 435.5, filed Jul. 9, 2003, and European Application No. 03 015 741.6, filed Jul. 10, 2003, each of which is hereby incorporated by reference in its entirety.

### FIELD OF THE INVENTION

This invention relates to a pharmaceutical composition comprising meloxicam or a pharmaceutically acceptable salt thereof and at least a second pharmaceutically active compound selected from the group consisting of antacids, sedatives, and central nervous system stimulants. Furthermore, this invention relates to an oral pharmaceutical dosage form comprising such a composition. A further objective of this invention is related to the use of the composition and the oral pharmaceutical dosage form. In addition, this invention relates to the use of meloxicam and at least a second pharmaceutically active compound selected from the group consisting of antacids, sedatives, and central nervous system stimulants for the manufacture of such an oral pharmaceutical dosage form. Furthermore, this invention relates to a method of treating or alleviating of an inflammatory disease, symptoms of an inflammatory disease, headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction, and/or cold and various symptoms of cold such as sore throat, chill, pyrexia, headache, arthralgia, and muscle pain.

### BACKGROUND OF THE INVENTION

Non-steroidal anti-inflammatory drugs (NSAIDs) are a kind of medicine that have been used in a broad range of treatment for many years for treating many patients with various diseases. Existing NSAIDs are cyclooxygenase (COX) inhibitors which nonspecifically inhibit cyclooxygenase (COX), a rate-limiting enzyme for biosynthesis of prostaglandin (PG) from arachidonic acid. Inhibition of COX contributes to anti-inflammatory, analgesic, and antipyretic effects by inhibiting production of PGE₂, on the other hand, they also cause adverse drug reactions such as digestive disorders and renal disorders. Incidentally, COX includes two types of isoforms, i.e., COX-1 and COX-2. COX-1 is constitutively (a certain amount of protein is developed regardless of proliferation or environmental changes) developed in most of the organs such as stomach and kidneys. Furthermore it has become evident that COX-2 is induced by various inflammatory mediators or endotoxin in local inflammatory areas.

Meloxicam, a recently developed selective COX-2 inhibitor, is increasingly being used for its contribution to reduce the risks of undesirable adverse drug reactions occurring in the gastrointestinal tract, and superior anti-inflammatory, analgesic, and antipyretic effects. However, a selective COX-2 inhibitor still may cause some side-effects in the digestive system by oral administration such as stomach indisposition and stomachache. Thus, oral pharmaceutical compositions with strengthened expression of efficacy and superior safety by potentiating anti-inflammatory, analgesic, and antipyretic effects, while alleviating side effects such as gastrointestinal disorders are desired.

A pharmaceutical composition comprising a COX-2 inhibitor and opioid analgesics as a method for potentiating the expression of efficacy of meloxicam is disclosed in the specification of the international publication WO 99/13799. A pharmacological composition comprising a combination of morphine and meloxicam in the ratio of 1:10 is disclosed therein. However, such a composition is unfavorable for safety reasons since opioid may cause undesirable side-effects.

A pharmaceutical composition comprising a selective COX-2 inhibitor and a 5HT_{1B/1D} receptor agonist as effective for treatment and/or prevention of migraine is disclosed in the specification of the international publication WO 00/25779.

A combination of an inhibitor of nitric oxide synthase induced for treatment of inflammation and inflammatory diseases and a COX-2 inhibitor as effective for treatment of inflammation-related diseases is disclosed in the specification of the international publication WO 99/18960.

A combination of a COX-2 inhibitor and a leukotriene B4 receptor antagonist as effective for treatment of inflammation and inflammation-related diseases is disclosed in the specification of the international publication WO 96/41645.

A combination of a COX-2 inhibitor and a 5-lipoxygenase inhibitor as effective for treatment of inflammation and inflammation-related diseases is disclosed in the specification of the international publication WO 96/41626.

A composition comprising a combination of a COX-2 inhibitor and a leukotriene A4 hydrolase inhibitor as effective for treatment of inflammation and inflammation-related diseases is disclosed in the specification of the international publication WO 96/41625.

A pharmaceutical composition comprising selective COX-2 inhibiting NSAIDs and antiallergics or antihistamines as effective for the treatment of rhinitis is disclosed in the Publication of the Japanese Patent Application JP2001-247481A. In Example 2 of that application, a composition comprising meloxicam, epinastine hydrochloride, phenylpropanolamine hydrochloride, and lysozyme chloride in tablet form is disclosed. However, an antacid is not included in the composition.

A solid pharmaceutical preparation for internal use with promoted absorption of anti-inflammatory drugs of the oxicam group selected from chlortenoxicam, tenoxicam, or piroxicam by combining with antacids is disclosed in the Japanese Patent Application JP2-906528A. However, chlortenoxicam (lornoxicam), tenoxicam, and piroxicam mentioned in the patent publication are all COX inhibitors which nonspecifically inhibit cyclooxygenase (COX). Meloxicam, which is a selective COX-2 inhibitor, is not exemplified therein.

One aim of the present invention is to further improve the safety profile of meloxicam with respect to possible side effects.

Another aim of the present invention is to provide a pharmaceutical composition and an oral pharmaceutical dosage form each comprising meloxicam and each having improved anti-inflammatory, analgesic, and antipyretic effects.

The invention also aims to provide pharmaceutical compositions and oral pharmaceutical dosage forms with improved efficacy and safety.

A further aim of the present invention is to provide a method of treating or alleviating of inflammatory diseases, headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction, and the like.

### DETAILED DESCRIPTION OF THE INVENTION

This invention relates to new pharmaceutical compositions comprising meloxicam or a pharmaceutically acceptable salt thereof and at least a second pharmaceutically active compound selected from the group consisting of antacids, sedatives, and central nervous system stimulants. Preferably meloxicam or the salt thereof is present in an effective amount allowing itself to exert anti-inflammatory, analgesic, and antipyretic effects.

The composition of the present invention allows a reinforcement of the therapeutic effects of meloxicam such as analgesic, anti-inflammatory, and antipyretic effects, while providing good bioavailability.

Especially by choosing an antacid as the second pharmaceutically active compound, the possibility of side effects in the digestive system by oral administration such as stomach indisposition and stomachache can be decreased. The composition of the present invention has superior safety and is therefore advantageously suitable for the manufacture of a non-prescription drug.

An advantage of the present invention when choosing a sedative and/or a central nervous system stimulant as the second pharmaceutically active compound is that the composition of the invention allows a reinforcement of the therapeutic effects such as analgesic, anti-inflammatory, and antipyretic effects without the necessity of increasing the dose of meloxicam. Therefore, it is possible to provide pharmaceutical compositions and oral pharmaceutical dosage forms with improved efficacy and safety. Thus the composition of the present invention is especially suitable for the manufacture of a non-prescription drug.

Meloxicam is a known selective COX-2 inhibitor which belongs to the acid enolcarboxamide (oxicam) type of nonsteroidal anti-inflammatory drugs (NSAIDs). The compound (4-hydroxy-2-methyl-N-(5-methyl-2-thiazolyl)-2H-1,2-benzothiazine-3-carboxamide 1,1-dioxide) is described in EP 0 002 482 B1 and U.S. Pat. No. 4,233,299.

The invention may employ either meloxicam itself or a pharmaceutically acceptable salt thereof. The pharmaceutically acceptable salt of meloxicam includes sodium salt, potassium salt, ammonium salt, meglumine salt, TRIS salt, and salts of meloxicam with a basic amino acid as examples. Various salts of meloxicam are described in EP 0 002 482 B1, U.S. Pat. No. 4,233,299, and WO 99/49867.

According to a first embodiment of this invention, the pharmaceutical composition comprises meloxicam or a pharmaceutically acceptable salt thereof and at least one antacid.

According to a second embodiment of this invention, the pharmaceutical composition comprises meloxicam or a pharmaceutically acceptable salt thereof and at least one sedative. In addition to meloxicam and a sedative mentioned above, the compositions according to the second embodiment of the present invention may also include other pharmacologically active substances such as an antacid.

According to a third embodiment of this invention, the pharmaceutical composition comprises meloxicam or a pharmaceutically acceptable salt thereof and at least one central nervous system stimulant.

In addition to meloxicam and a central nervous system stimulant mentioned above, the compositions according to the third embodiment of the present invention may also include other pharmacologically active substances such as an antacid and/or a sedative. According to a preferred modification of the third embodiment of this invention, the pharmaceutical composition comprises meloxicam or a pharmaceutically acceptable salt thereof and at least one central nervous system stimulant and at least one sedative. In addition to meloxicam and central nervous system stimulant and optionally a sedative as mentioned above, the pharmaceutical composition of the present invention may also include other pharmacologically active substances such as an antacid.

The term “antacid” according to the invention encompass two types. A first type neutralizes stomach acid. A second type controls the stomach acid secretion. According to this invention, the first type with stomach acid neutralization effect is preferred.

Preferably, the antacid is selected from the group consisting of aminoacetic acid, magnesium silicate, synthetic aluminum silicate, hydrotalcite, magnesium oxide, dihydroxy aluminum aminoacetate, aluminum hydroxide gel, dried aluminum hydroxide gel, aluminum hydroxide-magnesium carbonate co-dried gel, aluminum hydroxide-sodium bicarbonate coprecipitate, aluminum hydroxide-calcium carbonate-magnesium carbonate coprecipitate, magnesium hydroxide-potassium aluminum sulfate coprecipitate, magnesium carbonate, magnesium aluminometasilicate, magnesium aluminosilicate, aluminum magnesium hydroxide, magnesium hydroxide, sodium hydrogen carbonate, precipitated calcium carbonate, anhydrous dibasic calcium phosphate, and calcium hydrogen phosphate.

Examples of antacids which are most preferred are selected from the group consisting of aminoacetic acid, synthetic aluminum silicate, hydrotalcite, magnesium oxide, dihydroxy aluminum aminoacetate, aluminum hydroxide gel, dried aluminum hydroxide gel, aluminum hydroxide-sodium bicarbonate coprecipitate, magnesium carbonate, magnesium aluminometasilicate, magnesium hydroxide, sodium hydrogen carbonate and calcium hydrogen phosphate.

Although not preferred, the composition according to this invention may also comprise an antacid which controls the stomach acid secretion. Examples of such antacids are histamine H2 receptor antagonists such as cimetidine, famotidine, ranitidine hydrochloride, roxatidine acetate hydrochloride, nizatidine, and lafutidine, proton pump inhibitors such as omeprazole, lansoprazole, leminoprazol, pantoprazol, and rabeprazole sodium, and selective muscarine receptor antagonists such as tiquizium bromide and pirenzepine hydrochloride.

Preferably, the sedative is selected from the group consisting of allylisopropylacetylurea ((2-isopropyl-4-pentenoyl)urea) and bromvalerylurea ((2-bromo-3-methylbutyryl)urea).

Preferably, the central nervous system stimulant is selected from the group consisting of caffeine (3,7-dihydro-1,3,7-trimethyl-1H-purine-2,6-dione monohydrate), anhydrous caffeine (3,7-dihydro-1,3,7-trimethyl-1H-purine-2,6-dione) and a salt complex of caffeine and sodium benzoate (caffeine and sodium benzoate). Also combinations of two or more central nervous system stimulants may be used.

According to a preferred embodiment, the pharmaceutical composition according to the invention further comprises at least one pharmaceutically acceptable carrier and/or excipient. Suitable carriers and excipients are known to the one skilled in the art and are described, for example, in Japanese Pharmaceutical Excipients Directory 2000 (edited by Japan Pharmaceutical Excipients Council, issued by Yakuji Nippo, Ltd.) which is hereby incorporated by reference. Examples of suitable carriers and/or excipients are lactose, sucrose, glucose, mannitol, xylitol, corn starch, potato starch, wheat starch, rice starch, tapioca starch, sodium carboxymethyl starch, microcrystalline cellulose, ethylcellulose, hydroxypropylmethylcellulose, low substituted hydroxypropylcellulose, hydroxypropylcellulose, croscarmellose sodium, carmellose, carmellose potassium, carmellose calcium, carmellose sodium, polyvinylpyrrolidone, propyleneglycol, polyethyleneglycol, glycerol, vegetable oils, waxes, crospovidone, agar, light anhydrous silicic acid, magnesium stearate, talc, titanium oxide, acacia, sodium alginate, ethanol, and purified water.

In addition, the present invention relates to a pharmaceutical dosage form which comprises a pharmaceutical composition according to this invention.

The amount of meloxicam used for the oral pharmaceutical dosage form described in the invention is preferably in the range from 1 mg to 30 mg, more preferably in the range from 2.5 mg to 15 mg, and most preferably in the range from 5 mg to 10 mg. These amounts correspond to the preferred dosage ranges with respect to an adult and once daily given dose.

According to the first embodiment of this invention, the at least one second pharmaceutically active compound is selected from the group consisting of antacids. Although the amount of the antacid in the oral pharmaceutical dosage form according to the present invention may be varied depending on the type of the antacid, it preferably lies in the range from 10 mg to 7000 mg, and more preferably in the range from 16 mg to 4000 mg. These amounts correspond to the preferred dose given daily to an adult.

With respect to the preferred antacids, the amount of the respective antacid in the oral pharmaceutical dosage form according to this invention lies preferably in the range from 30 mg to 900 mg for aminoacetic acid and aluminum hydroxide-sodium bicarbonate coprecipitate, in the range from 100 to 3000 mg for magnesium silicate, synthetic aluminum silicate, and aluminum hydroxide-magnesium carbonate co-dried gel, in the range from 133 mg to 4000 mg for hydrotalcite, in the range from 16 mg to 500 mg for magnesium oxide, in the range from 50 mg to 1500 mg for dihydroxy aluminum aminoacetate, aluminum hydroxide-calcium carbonate-magnesium carbonate coprecipitate, magnesium aluminometasilicate, precipitated calcium carbonate, and calcium hydrogen phosphate, in the range from 33 mg to 1000 mg for aluminum hydroxide gel and dried aluminum hydroxide gel as dried aluminum hydroxide gel, in the range from 60 to 1800 mg for magnesium hydroxide-potassium aluminum sulfate coprecipitate, in the range from 66 mg to 2000 mg for magnesium carbonate, magnesium aluminosilicate, and aluminum magnesium hydroxide, in the range from 40 mg to 1200 mg for magnesium hydroxide and anhydrous dibasic calcium phosphate, and in the range from 83 mg to 2500 mg for sodium hydrogen carbonate. The given amounts correspond to the preferred dose of the respective antacid given daily to an adult according to this invention.

According to the second embodiment of this invention, the at least one second pharmaceutically active compound is selected from the group consisting of sedatives. Although the amount of the sedative in the oral pharmaceutical dosage form according to the present invention may be varied depending on the type of the sedative, it preferably lies in the range from 15 mg to 1200 mg, and more preferably in the range from 30 mg to 600 mg. These amounts correspond to the preferred dose given daily to an adult.

With respect to the preferred sedatives, the amount of the respective sedative in the oral pharmaceutical dosage form according to this invention lies preferably in the range from 30 mg to 180 mg for allylisopropylacetylurea, and in the range from 100 mg to 600 mg for bromvalerylurea. The given amounts correspond to the preferred dose of the respective sedative given daily to an adult according to this invention.

According to the third embodiment of this invention the at least one second pharmaceutically active compound is selected from the group consisting of central nervous system stimulants. Although the amount of the central nervous system stimulant in the oral pharmaceutical dosage form according to the present invention may be varied depending on the type of the central nervous system stimulant, it preferably lies in the range from 15 mg to 1200 mg, and more preferably in the range from 30 mg to 300 mg. These amounts correspond to the preferred dose given daily to an adult.

With respect to the preferred central nervous system stimulants, the amount of the respective central nervous system stimulant in the oral pharmaceutical dosage form according to this invention lies preferably in the range from 60 mg to 300 mg for caffeine and sodium benzoate, and in the range from 30 mg to 250 mg for caffeine and/or anhydrous caffeine. The given amounts correspond to the preferred dose of the respective central nervous system stimulant given daily to an adult according to this invention.

In case the oral pharmaceutical dosage form according to the third embodiment additionally comprises a sedative, the amount of the sedative in the oral pharmaceutical dosage form may be varied depending on the type of the sedative. It preferably lies in the range from 15 mg to 1200 mg, and more preferably in the range from 30 mg to 600 mg. These amounts correspond to the preferred dose given daily to an adult.

With respect to the preferred sedatives, the amount of the respective sedative in such a oral pharmaceutical dosage form lies preferably in the range from 30 mg to 180 mg for allylisopropylacetylurea, and in the range from 100 mg to 600 mg for bromvalerylurea. The given amounts correspond to the preferred dose of the respective sedative given daily to an adult according to this invention.

The oral pharmaceutical dosage form of the invention may be orally given in divided doses, however, it is preferable to be orally given once a day. Dose adjustment of meloxicam and the at least one second pharmaceutically active compound may reflect age, body weight, and manifesting symptoms.

The oral pharmaceutical dosage form described in the present invention comprises tablets, granules, fine granules, powders, capsules, caplets, soft capsules, pills, oral solutions, syrups, dry syrups, chewable tablets, troches, forming tablets, drops, suspensions, and fast dissolving tablets. Any of these formulations may be prepared using regular methods, and, in addition to the aforementioned components, any additives in common use may be used upon preparation of these formulations, if necessary. In addition, preparations formed into microparticles such as microcapsules, nanocapsules, microspheres, nanospheres, and liposomes may also be included in the aforementioned formulations.

In addition, further components of the oral pharmaceutical dosage form and the formulation of all ingredients are preferably chosen in view of the desired mechanical, chemical, and biological stability, release rate, masking of the taste, visual appearance, etc.

For example, the pharmaceutically active substances, i.e., meloxicam or a pharmaceutically salt thereof and the second pharmaceutically active compound, can be dispensed in separate granules, multi-layer granules, multi-layer tablets or dry coated tablets, tablets of separated granules, microcapsules, etc. Coating preparations such as sugarcoated tablets, film coating tablets, coating granule, foaming pharmaceutical preparation can be used as well as chewable preparations, fast dissolving preparations, matrix preparations, together with comminutions, solid solutions, etc. These methods can also be combined.

According to a preferred embodiment, the oral dosage form is a combination of a first dosage form comprising meloxicam or a pharmaceutically acceptable salt thereof and a second dosage form comprising the at least second pharmaceutically active compound. Preferably the first dosage form releases the active ingredients faster than the second dosage form. The first dosage form may further comprise the second pharmaceutically active compound and optionally further active ingredients. The second dosage form may comprise further active ingredients. Preferably the second dosage form does not comprise meloxicam.

For example, the dosage form is a two layer tablet wherein the first layer comprises meloxicam or a pharmaceutical acceptable salt thereof and optionally a second pharmaceutically active compound, as for example a sedative or a central nervous system stimulant, and optionally further active ingredients and pharmaceutically acceptable carriers and/or excipients. The second layer comprises the second pharmaceutically active compound, as, for example, a sedative or a central nervous system stimulant, and optionally further active ingredients and pharmaceutically acceptable carriers and/or excipients, whereby the second layer has slow release properties compared with the first layer.

According to a further example, the dosage form is a capsule comprising two kinds of granules. The first kind of granules comprise meloxicam or a pharmaceutical acceptable salt thereof and optionally a second pharmaceutically active compound, as for example a sedative or a central nervous system stimulant, and optionally further active ingredients and pharmaceutically acceptable carriers and/or excipients. The second kind of granules comprise the second pharmaceutically active compound, as, for example, a sedative or a central nervous system stimulant, and optionally further active ingredients and pharmaceutically acceptable carriers and/or excipients, whereby the second kind of granules have slow release properties compared with the first kind of granules.

These formulations may be prepared using regular methods by adding generally available pharmaceutical additives such as excipients, binders, disintegrators, lubricants, coating agents, sugar coating agents, plasticizers, antifoaming agents, polish, foaming agents, antistatic agents, desiccants, surfactant, solubilizer, buffer agents, resolvents, solubilizing agents, solvents, diluents, stabilizers, emulsifying agents, suspensions, suspending agents, dispersing agents, isotonizing agents, adsorbents, reducing agents, antioxidants, wetting agents, wet modifiers, fillers, extenders, adhesives, viscous agent, softeners, pH modifiers, antiseptics, preservatives, sweetening agents, corrigents, refrigerative agents, flavoring agents, perfumes, fragrances, and coloring matters to the pharmacologically active compounds. Examples of such additives are described in Japanese Pharmaceutical Excipients Directory 2000 (edited by Japan Pharmaceutical Excipients Council, issued by Yakuji Nippo, Ltd.).

These preparations are preferably manufactured by adding pharmaceutical additives to the pharmacologically active compounds.

The pharmaceutical compositions and dosage forms according to the first embodiment of this invention are advantageously useful as analgesics, antipyretics, and/or antacids.

The pharmaceutical compositions and dosage forms according to the second embodiment of this invention are advantageously useful as analgesics, antipyretics, and/or sedatives.

The pharmaceutical compositions and dosage forms according to the third embodiment of this invention are advantageously useful as analgesics, antipyretics, and/or central nervous system stimulants.

The invention relates to the use of the pharmaceutical composition and of the oral pharmaceutical dosage form, both as described hereinbefore, for the treatment and alleviation of inflammatory diseases, including various symptoms thereof.

The pharmaceutical compositions and dosage forms of the invention are effective for the treatment and alleviation of headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction, and the like.

Moreover, the pharmaceutical compositions and dosage forms of the invention are also effective for alleviation of various symptoms of cold such as sore throat, chill, pyrexia, headache, arthralgia, and muscle pain.

Furthermore, this invention relates to the use of a pharmaceutical composition as described hereinbefore for the manufacture of a medicament for the prevention or alleviation of an inflammatory disease, including various symptoms thereof, and/or headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction, and/or cold and various symptoms of cold such as sore throat, chill, pyrexia, headache, arthralgia, and muscle pain.

In addition, this invention relates to the use of meloxicam or a pharmaceutically acceptable salt thereof for the manufacture of an oral pharmaceutical dosage form according to this invention.

Thus, the invention also relates to the use of a pharmaceutically active compound selected from the group consisting of antacids, sedatives, and central nervous system stimulants, for the manufacture of an oral pharmaceutical dosage form according to this invention.

Consequently, this invention further relates to a method of treating or alleviating of an inflammatory disease, symptoms of an inflammatory disease, including various symptoms thereof, and/or headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction, and/or cold and various symptoms of cold such as sore throat, chill, pyrexia, headache, arthralgia, and muscle pain, in a patient in need of such treatment, which comprises orally administering to the patient a pharmaceutical composition according to this invention.

The patient to be treated according to this invention is a mammal, preferably a human.

The preferred daily dose orally administered to the patient according to this invention is in the range of 1 mg to 30 mg meloxicam, more preferably 2.5 mg to 15 mg meloxicam or a salt thereof, and
(a) in case the second pharmaceutically active compound is an antacid, an amount of 10 mg to 7000 mg, more preferably 16 mg to 4000 mg, of an antacid;
(b) in case the second pharmaceutically active compound is a sedative, an amount of 15 mg to 1200 mg, more preferably 30 mg to 600 mg, of a sedative; and
(c) in case the second pharmaceutically active compound is a central nervous system stimulant, an amount of 15 mg to 1200 mg, more preferably 30 mg to 300 mg, of a central nervous system stimulant.

In case a sedative is chosen as a further active ingredient, the preferred daily dose orally administered to the patient according to this invention is in the range of 15 mg to 1200 mg, more preferably 30 mg to 600 mg, of the sedative.

The preferred doses regarding the various second pharmaceutically active compounds were specified above. Therefore, the amount of the dosage form to be taken by a patient per day, i.e., the number of tablets, capsules, caplets, troches, etc., or the amount of granules, syrup, solution, suspension, etc., e.g., measured in grams or milliliters, is such that the above specified preferred daily dose is achieved.

Meloxicam or a pharmaceutically acceptable salt thereof and the at least one pharmaceutically active compound are preferably combined in a single oral dosage form as described above. Both meloxicam or a salt thereof and the second pharmaceutically active compound may also be simultaneously administered in two separate oral dosage forms, one containing meloxicam or a salt thereof and the other containing the second pharmaceutically active compound.

The compositions and dosage forms of the present invention are explained by the following Examples which however do not limit the scope of the present invention. Examples 1.1 to 1.6 illustrate the first embodiment of the present invention, Examples 2.1 to 2.7 illustrate the second embodiment of the present invention, and Examples 3.1 to 3.7 illustrate the third embodiment of the present invention.

### EXAMPLES

### Example 1.1

### Tablet

The following ingredients are homogeneously mixed. The resulted mixed particles are compressed with a mold to prepare tablets at 300 mg each.

| | |
|---|---|
| Meloxicam | 15 g |
| Aminoacetic acid | 600 g |
| Lactose | 585 g |
| Microcrystalline cellulose | 564 g |
| Light anhydrous silicic acid | 18 g |
| Magnesium stearate | 10 g |
| Talc | 8 g |

### Example 1.2

### Powder

The following ingredients are homogeneously mixed. The resulted mixed particles are divided into portions of 800 mg to prepare powder compositions.

| | |
|---|---|
| Meloxicam | 15 g |
| Dried aluminum hydroxide gel | 600 g |
| Corn starch | 469 g |
| Lactose | 500 g |
| Magnesium stearate | 16 g |

### Example 1.3

### Two Layer Tablet

The following ingredients of the layer A and the layer B are processed through a regular method to provide mixed particles, respectively, and the particles are compressed to form two layer tablets at 250 mg (layer A: 100 mg and layer B: 150 mg) each.

| | |
|---|---|
| Layer A: | |
| Meloxicam | 45 g |
| Lactose | 273 g |
| Microcrystalline cellulose | 270 g |
| Light anhydrous silicic acid | 6 g |
| Talc | 3 g |
| Magnesium stearate | 3 g |
| Layer B: | |
| Magnesium oxide | 300 g |
| Lactose | 282 g |
| Microcrystalline cellulose | 300 g |
| Light anhydrous silicic acid | 9 g |
| Talc | 3 g |
| Magnesium stearate | 6 g |

### Example 1.4

### Granules

The following ingredients are prepared as granules through a regular method to prepare mixed particles, and packed to give an amount of 1400 mg per one pack of granules.

| | |
|---|---|
| Meloxicam | 15 g |
| Dihydroxy aluminum aminoacetate | 200 g |
| Magnesium carbonate | 400 g |
| Calcium carboxymethylcellulose | 400 g |
| Mannitol | 1100 g |
| Sodium lauryl sulfate | 10 g |
| Corn starch | 649 g |
| Aspartame | 12 g |
| Acesulfame potassium | 12 g |
| Fragrant materials | 2 g |

### Example 1.5

### Film-Coated Tablet

The following ingredients are processed through a regular method to provide mixed particles, and the particles are compressed to form tablets at 240 mg each.

| | |
|---|---|
| Meloxicam | 45 g |
| Synthetic aluminum silicate | 720 g |
| Lactose | 1320 g |
| Microcrystalline cellulose | 900 g |
| Corn starch | 753 g |
| Low substituted hydroxypropylcellulose | 270 g |
| Hydroxypropylcellulose | 180 g |
| Light anhydrous silicic acid | 90 g |
| Talc | 24 g |
| Magnesium stearate | 18 g |

Subsequently, the tablets are transferred into a coating pan, and coated using a coating solution, containing the equal volume mixture of ethyl alcohol, containing 5% weight/volume of hydroxypropylmethylcellulose, and purified water to increase in weight/volume by 10 mg per one tablet. Thus film-coated tablets are prepared.

### Example 1.6

### Capsules

The following ingredients are prepared as granules through regular methods, and capsule-filled to give an amount of 150 mg per one capsule.

| | |
|---|---|
| Meloxicam | 60 g |
| Magnesium aluminometasilicate | 1280 g |
| Microcrystalline cellulose | 400 g |
| Lactose | 260 g |
| Low substituted hydroxypropylcellulose | 200 g |
| Corn starch | 200 g |

### Example 2.1

### Tablet

The following ingredients are homogeneously mixed. The resulted mixed particles are compressed with a mold to prepare tablets at 240 mg each.

| | |
|---|---|
| Meloxicam | 15 g |
| Allylisopropylacetylurea | 120 g |
| Lactose | 409 g |
| Microcrystalline cellulose | 388 g |
| Light anhydrous silicic acid | 14 g |
| Magnesium stearate | 8 g |
| Talc | 6 g |

### Example 2.2

### Powder

The following ingredients are homogeneously mixed. The resulted mixed particles are divided into portions of 800 mg to prepare powder compositions.

| | |
|---|---|
| Meloxicam | 15 g |
| Bromvalerylurea | 400 g |
| Corn starch | 569 g |
| Lactose | 576 g |
| Magnesium stearate | 40 g |

### Example 2.3

### Two Layer Tablet

The following ingredients of the layer A and the layer B are processed through a regular method to provide mixed particles, respectively, and the particles are compressed to form two layer tablets at 200 mg (layer A: 100 mg and layer B 100 mg) each.

| | |
|---|---|
| Layer A: | |
| Meloxicam | 45 g |
| Allylisopropylacetylurea | 360 g |
| Lactose | 381 g |
| Microcrystalline cellulose | 390 g |
| Light anhydrous silicic acid | 12 g |
| Talc | 6 g |
| Magnesium stearate | 6 g |
| Layer B: | |
| Allylisopropylacetylurea | 720 g |
| Lactose | 228 g |
| Hydroxypropylmethylcellulose 2208 | 60 g |
| Hydrogenated oil | 60 g |
| Stearic acid | 60 g |
| Glycerol esters of fatty acids | 60 g |
| Magnesium stearate | 12 g |

### Example 2.4

### Granules

The following ingredients are prepared as granules through a regular method to prepare mixed particles, and packed to give an amount of 1000 mg per one pack of granules.

| | |
|---|---|
| Meloxicam | 15 g |
| Bromvalerylurea | 400 g |
| Calcium carboxymethylcellulose | 120 g |
| Mannitol | 1100 g |
| Corn starch | 343 g |
| Aspartame | 10 g |
| Acesulfame potassium | 10 g |
| Fragrant materials | 2 g |

### Example 2.5

### Film-Coated Tablet

The following ingredients are processed through a regular method to provide mixed particles, and the particles are compressed to form tablets at 240 mg each.

| | |
|---|---|
| Meloxicam | 45 g |
| Allylisopropylacetylurea | 540 g |
| Lactose | 1314 g |
| Microcrystalline cellulose | 1080 g |
| Corn starch | 747 g |
| Low substituted hydroxypropylcellulose | 270 g |
| Hydroxypropylcellulose | 180 g |
| Light anhydrous silicic acid | 90 g |
| Talc | 36 g |
| Magnesium stearate | 18 g |

Subsequently, the tablets are transferred into a coating pan, and coated using a coating solution, containing the equal volume mixture of ethyl alcohol, containing 5% weight/volume of hydroxypropylmethylcellulose, and purified water to increase in weight/volume by 10 mg per one tablet. Thus film-coated tablets are prepared.

### Example 2.6

### Capsules

The following ingredients are prepared as fast release granules and slow release granules through regular methods, and capsule-filled to give an amount of 225 mg (fast release granules: 75 mg, slow release granules: 150 mg) per one capsule.

| | |
|---|---|
| Fast release granules | |
| Meloxicam | 60 g |
| Allylisopropylacetylurea | 480 g |
| Microcrystalline cellulose | 660 g |
| Slow release granules | |
| Allylisopropylacetylurea | 960 g |
| Fumaric acid | 240 g |
| Hydrogenated oil | 240 g |
| Microcrystalline cellulose | 744 g |
| Ethylcellulose (coating layer) | 160 g |
| Glycerol esters of fatty acids (coating layer) | 44 g |
| Talc (coating layer) | 12 g |

### Example 2.7

### Capsules

The following ingredients are prepared as fast release granules and slow release granules through regular methods, and capsule-filled to give an amount of 225 mg (fast release granules: 75 mg and slow release granules: 150 mg) per one capsule.

| | |
|---|---|
| Fast release granules | |
| Meloxicam | 60 g |
| Allylisopropylacetylurea | 480 g |
| Microcrystalline cellulose | 660 g |
| Slow release granules | |
| Allylisopropylacetylurea | 960 g |
| Fumaric acid | 240 g |
| Microcrystalline cellulose | 720 g |
| Methacrylic acid copolymer S (coating layer) | 432 g |
| Glycerol esters of fatty acids (coating layer) | 32 g |
| Talc (coating layer) | 16 g |

### Example 3.1

### Tablet

The following ingredients are homogeneously mixed. The resulted mixed particles are compressed with a mold to prepare tablets at 240 mg each.

| | |
|---|---|
| Meloxicam | 15 g |
| Anhydrous caffeine | 240 g |
| Allylisopropylacetylurea | 120 g |
| Lactose | 289 g |
| Microcrystalline cellulose | 268 g |
| Light anhydrous silicic acid | 14 g |
| Magnesium stearate | 8 g |
| Talc | 6 g |

### Example 3.2

### Powder

The following ingredients are homogeneously mixed. The resulted mixed particles are divided into portions of 800 mg to prepare powder compositions.

| | |
|---|---|
| Meloxicam | 15 g |
| Caffeine and sodium benzoate | 300 g |
| Corn starch | 617 g |
| Lactose | 628 g |
| Magnesium stearate | 40 g |

### Example 3.3

### Two Layer Tablet

The following ingredients of the layer A and the layer B are processed through a regular method to provide mixed particles, respectively, and the particles are compressed to form two layer tablets at 220 mg (layer A: 100 mg and layer B: 120 mg) each.

| | |
|---|---|
| Layer A: | |
| Meloxicam | 45 g |
| Anhydrous caffeine | 480 g |
| Lactose | 309 g |
| Microcrystalline cellulose | 330 g |
| Light anhydrous silicic acid | 12 g |
| Sodium lauryl sulfate | 12 g |
| Talc | 6 g |
| Magnesium stearate | 6 g |
| Layer B: | |
| Anhydrous caffeine | 1020 g |
| Lactose | 168 g |
| Hydroxypropylmethylcellulose 2208 | 60 g |
| Hydrogenated oil | 60 g |
| Stearic acid | 60 g |
| Glycerol esters of fatty acids | 60 g |
| Magnesium stearate | 12 g |

### Example 3.4

### Granules

The following ingredients are prepared as granules through a regular method to prepare mixed particles, and packed to give an amount of 1000 mg per one pack of granules.

| | |
|---|---|
| Meloxicam | 15 g |
| Caffeine | 240 g |
| Calcium carboxymethylcellulose | 120 g |
| Mannitol | 1260 g |
| Corn starch | 343 g |
| Aspartame | 10 g |
| Acesulfame potassium | 10 g |
| Fragrant materials | 2 g |

### Example 3.5

### Film-Coated Tablet

The following ingredients are processed through a regular method to provide mixed particles, and the particles are compressed to form tablets at 240 mg each.

| | |
|---|---|
| Meloxicam | 45 g |
| Caffeine and sodium benzoate | 1350 g |
| Lactose | 504 g |
| Microcrystalline cellulose | 1080 g |
| Corn starch | 747 g |
| Low substituted hydroxypropylcellulose | 270 g |
| Hydroxypropylcellulose | 180 g |
| Light anhydrous silicic acid | 90 g |
| Talc | 36 g |
| Magnesium stearate | 18 g |

Subsequently, the tablets are transferred into a coating pan, and coated using a coating solution, containing the equal volume mixture of ethyl alcohol, containing 5% weight/volume of hydroxypropylmethylcellulose, and purified water to increase in weight/volume by 10 mg per one tablet. Thus film-coated tablets are prepared.

### Example 3.6

### Capsules

The following ingredients are prepared as fast release granules and slow release granules through regular methods, and capsule-filled to give an amount of 225 mg (fast release granules: 75 mg and slow release granules: 150 mg) per one capsule.

| | |
|---|---|
| Fast release granules | |
| Meloxicam | 60 g |
| Anhydrous caffeine | 640 g |
| Microcrystalline cellulose | 500 g |
| Slow release granules | |
| Anhydrous caffeine | 1280 g |
| Fumaric acid | 320 g |
| Microcrystalline cellulose | 584 g |
| Ethylcellulose (coating layer) | 128 g |
| Hydroxypropylmethylcellulose (coating layer) | 32 g |
| Glycerol esters of fatty acids (coating layer) | 44 g |
| Talc (coating layer) | 12 g |

### Example 3.7

### Capsules

The following ingredients are prepared as fast release granules and slow release granules through regular methods, and capsule-filled to give an amount of 225 mg (fast release granules: 75 mg and slow release granules: 150 mg) per one capsule.

| | |
|---|---|
| Fast release granules | |
| Meloxicam | 60 g |
| Anhydrous caffeine | 640 g |
| Microcrystalline cellulose | 500 g |
| Slow release granules | |
| Anhydrous caffeine | 1280 g |
| Fumaric acid | 240 g |
| Microcrystalline cellulose | 400 g |
| Methacrylic acid copolymer S (coating layer) | 432 g |
| Glycerol esters of fatty acids (coating layer) | 32 g |
| Talc (coating layer) | 16 g |

1. A pharmaceutical composition comprising:
(a) meloxicam or a pharmaceutically acceptable salt thereof; and
(b) one or more second pharmaceutically active compounds selected from antacids, sedatives, and central nervous system stimulants.

2. The pharmaceutical composition according to claim 1, wherein the one or more second pharmaceutically active compounds includes an antacid.

3. The pharmaceutical composition according to claim 1, wherein the one or more second pharmaceutically active compounds includes a sedative.

4. The pharmaceutical composition according to claim 1, wherein the one or more second pharmaceutically active compounds includes a central nervous system stimulant.

5. The pharmaceutical composition according to claim 1, wherein the one or more second pharmaceutically active compounds includes a central nervous system stimulant and a sedative.

6. The pharmaceutical composition according to claim 2, wherein the antacid is selected from aminoacetic acid, magnesium silicate, synthetic aluminum silicate, hydrotalcite, magnesium oxide, dihydroxy aluminum aminoacetate, aluminum hydroxide gel, dried aluminum hydroxide gel, aluminum hydroxide-magnesium carbonate co-dried gel, aluminum hydroxide-sodium bicarbonate coprecipitate, aluminum hydroxide-calcium carbonate-magnesium carbonate coprecipitate, magnesium hydroxide-potassium aluminum sulfate coprecipitate, magnesium carbonate, magnesium aluminometasilicate, magnesium aluminosilicate, aluminum magnesium hydroxide, magnesium hydroxide, sodium hydrogen carbonate, precipitated calcium carbonate, anhydrous dibasic calcium phosphate, and calcium hydrogen phosphate.

7. The pharmaceutical composition according to claim 2, wherein the antacid is selected from aminoacetic acid, synthetic aluminum silicate, hydrotalcite, magnesium oxide, dihydroxy aluminum aminoacetate, aluminum hydroxide gel, dried aluminum hydroxide gel, aluminum hydroxide-sodium bicarbonate coprecipitate, magnesium carbonate, magnesium aluminometasilicate, magnesium hydroxide, sodium hydrogen carbonate and calcium hydrogen phosphate.

8. The pharmaceutical composition according to claim 3, wherein the sedative is allylisopropylacetylurea or bromvalerylurea.

9. The pharmaceutical composition according to claim 5, wherein the sedative is allylisopropylacetylurea or bromvalerylurea.

10. The pharmaceutical composition according to claim 4, wherein the central nervous system stimulant is caffeine, anhydrous caffeine, or a salt complex of caffeine and sodium benzoate.

11. The pharmaceutical composition according to claim 5, wherein the central nervous system stimulant is caffeine, anhydrous caffeine, or a salt complex of caffeine and sodium benzoate.

12. The pharmaceutical composition according to claim 5, wherein the sedative is allylisopropylacetylurea or bromvalerylurea and the central nervous system stimulant is caffeine, anhydrous caffeine, or a salt complex of caffeine and sodium benzoate.

13. The pharmaceutical composition according to one of claims 1 to 12, wherein the meloxicam or a pharmaceutically acceptable salt thereof is the meloxicam meglumine salt.

14. The pharmaceutical composition according to one of claims 1 to 12, further comprising at least one pharmaceutically acceptable carrier and/or excipient.

15. The pharmaceutical composition according to claim 13, further comprising at least one pharmaceutically acceptable carrier and/or excipient.

16. The pharmaceutical composition according to one of claims 1 to 12, wherein the pharmaceutical composition is oral pharmaceutical dosage form and the amount of meloxicam or a pharmaceutically acceptable salt thereof is in the range of 1 mg to 30 mg.

17. The pharmaceutical composition according to one of claims 1 to 12, wherein the pharmaceutical composition is oral pharmaceutical dosage form and the amount of meloxicam or a pharmaceutically acceptable salt thereof is in the range of 1 mg to 30 mg and the amount of the one or more second pharmaceutically active compounds are (a) in the range from 10 mg to 7000 mg when the second pharmaceutically active compound is an antacid, and (b) in the range from 15 mg to 1200 mg when the second pharmaceutically active compound is a sedative or a central nervous system stimulant.

18. A method of treating or alleviating of an inflammatory disease, symptoms of an inflammatory disease, including various symptoms thereof, and/or headache, toothache, ache after tooth extraction, sore throat, otalgia, arthralgia, neuralgia, lumbago, myalgia, muscle stiffness of shoulder, pain of contusion, pain of fracture, pain of sprain, menstrual pain, traumatic pain, chill, exothermic reaction and/or cold and various symptoms of cold such as sore throat, chill, pyrexia, headache, arthralgia and muscle pain in a patient in need of such treatment, which comprises orally administering to the patient a pharmaceutical composition according to one of claims 1 to 12.
